(19) United States
(12) Patent Application Publication
(10) Pub. No.: US 2008/0226694 A1
(43) Pub. Date: Sep. 18, 2008

(54) Method for introducing superhydrophobic articles into the human body

(76) Inventors: Daniel Gelbart, Vancouver (CA); Samuel Victor Lichtenstein, Vancouver (CA)

Correspondence Address: DANIEL GELBART, 4706 DRUMMOND DR, VANCOUVER, BC, V6T-1B4, CA

(21) Appl. No.: 11/717,071

(22) Filed: Mar. 13, 2007

(51) Int. Cl. A61L 27/40

(52) U.S. Cl. 424/426

(57) ABSTRACT

An article to be inserted into the human body has a superhydrophobic surface. The superhydrophobic surface is coated with a water soluble thin but durable protective coat. One positioned inside the body the coating is rapidly dissolved by the blood or other fluids and exposes the superhydrophobic surface. To coat article the water based coating is mixed with a liquid capable of wetting the superhydrophobic surface but is still dissolvable or at least miscible in the coating. As an example, a glucose or sucrose solution in water is mixed with alcohol and used to coat the surface. After water and alcohol evaporation, a durable protective coat of sugar remains. After the coated article is inserted into the body, the coating is rapidly dissolved and absorbed by the body.

### FIELD OF INVENTION

The invention is in the medical field and of particular use in cardio-vascular medicine.

### BACKGROUND OF THE INVENTION

It is known that superhydrophobic coatings have benefits in medicine because of their tendency to repel any water based or water containing substance such as blood, tissue or bacterial growth. This is particularly important when a non-thrombogenic surface is needed in devices introduced into the human blood system. In order to achieve superhydrophobic behavior a very delicate and fine surface texture is required. Such a texture can be easily damaged by handling or even gentle contact with any solids, making it very difficult to insert such surfaces into the body without damage.

### SUMMARY OF THE INVENTION

According to the invention the sensitive superhydrophobic surface is coated with a thin water soluble and durable protective coat. Once positioned inside the body the coating is rapidly dissolved by the blood or other fluids and exposes the superhydrophobic surface. Unfortunately coatings that are very water soluble are also water based, and it nearly impossible to directly coat a superhydrophobic surface with a water based coating, as it will be strongly repelled. According to the invention the water based coating is mixed with a liquid capable of wetting the superhydrophobic surface but is still dissolvable or at least miscible in the coating. As an example, a glucose or sucrose solution in water is mixed with alcohol and used to coat the surface. After water and alcohol evaporate, a durable protective coat of sugar remains. After the coated article is inserted into the body, the coating is rapidly dissolved and absorbed by the body.

### BRIEF DESCRIPTION OF THE DRAWINGS

FIG. 1-A is a perspective view, including a magnified cross section, of a stent coated according to the invention in the non-expanded state.

FIG. 1-B is a perspective view, including a magnified cross section, of a stent coated according to the invention in the expanded state immediately after deployment.

FIG. 1-A is a perspective view, including a magnified cross section, of a stent coated according to the invention in the expanded state a short time after deployment.

### DESCRIPTION OF THE PREFERRED EMBODIMENT

The phenomena of superhydrophobicity is well known in nature, for example the way water rolls off the leaves of the lotus plant. The definition of a superhydrophobic surface is a surface forming a contact angle of more than 150 degrees with a drop of water. Such surfaces rely on a combination of surface texture and surface chemistry rather than just chemical surface energy. Without surface texturing it is difficult to get contact angles larger than 120 degrees even in fluorocarbons and silicones. With microscopic surface texturing, at the nanometer to micrometer level, contact angles as high as 178 degrees are possible. The textured surface can be polymeric (“polymer brushes”), inorganic (silicon dioxide) or even metallic. On metallic surfaces a very thin coat (typically a monolayer) of surface modifier is normally used. The most hydrophobic surfaces are covered with “bristles” having a diameter of a few nanometers and a length of a few hundred nanometers, making them extremely delicate and difficult to handle. The surfaces that are more durable exhibit lower contact angle, such as 160-170 degrees. It is known from medical experiments that for contact angles of 150 degrees or less there are no anti-thrombogenic benefits over a regular hydrophobic material such as fluorocarbon having contact angles of around 120 degrees. Because of this only the most superhydrophobic surfaces, with contact angles approaching 180 degrees are of the greatest interest. It is believed that on these surfaces a sub-micron air layer is created separating the liquid from the surface, even when fully submerged for extended periods of time. Such an air layer prevents any contact between liquids such as blood and the surface and prevents bacteria and tissue growth over the surface. These properties are very desirable particularly in objects inserted into the blood streams such as stents, artificial heart valves, artificial hearts, pacemakers, arterial closure devices and vascular grafts. They are also desirable in urinary stents and other implants that need to stay clean inside the body. Even if a material can be deposited temporarily on a superhydrophobic surface it will not be able to attach itself and will be washed away.

When introducing medical devices into the body there is a desire to minimize the size of the incision, therefore many devices are inserted in a folded or a compressed form. This is particularly true for percutaneous procedures, where the device is inserted at a point that could be far from the final deployment point in the body. The fine surface texture of the best superhydrophobic materials is not able to withstand pressure or even light contact by other objects, therefore can not be used in today's minimally invasive procedures. According to the invention the delicate surface is coated with a water soluble protective coating. The coating can be engineered to dissolve at the correct speed, so it does not dissolve during the introduction into the body but dissolves shortly after. The coating is made of a material compatible with the body, and even more desirable a coating of a sugar such as glucose or sucrose that is digested by the body. The thickness of the coating is typically in the range of 1-10 microns. The speed with which the coating dissolves in the body can be controlled by the choice of material, thickness, a secondary top coating or treatment. Example of treatments than can be applied to a sugar coating in order to slow down dissolution rate are adding a hydrophobic surface treatment to the sugar (such as a monolayer of fat) or partially caramelizing the coating by heating just the top surface to 150-200 degrees C. Dissolution time for a 5 um glucose or sucrose coating can be as short as a few seconds or as long as hours depending on surface treatment. The small amount of sugar (typically under one milligram) is absorbed in the body without any side effects, as a much larger amount of identical materials already exist in the body. For example, the blood contains many grams of glucose therefore another milligram coming from the dissolved coating has no effect.

In order to coat the superhydrophobic surface a strong wetting agent that is dissolvable, or at least miscible, with the water based solution is needed. Without such a wetting agent the water based coating will simply roll off the superhydrophobic surface. By the way of example, adding 30%-60% ethanol to a glucose or sucrose solution makes it fully and uniformly able to wet a metallic superhydrophobic coating. The amount of alcohol can be used to control the thickness of the coating. After alcohol and water have evaporated a glossy protective coat is clearly visible instead of the dull black appearance of the uncoated material. When contacting water or blood the coating is dissolved and repelled; the material returns to a dull black appearance. Since the anti-thrombogenic properties of such coatings are based on a physical effect rather than action of a drug, they are more desirable than drug-eluting coatings. There is also less chance of interaction between the coating and any drugs the patient is on.

As an example of the preferred embodiment the complete process of coating and deploying a stent will be described.

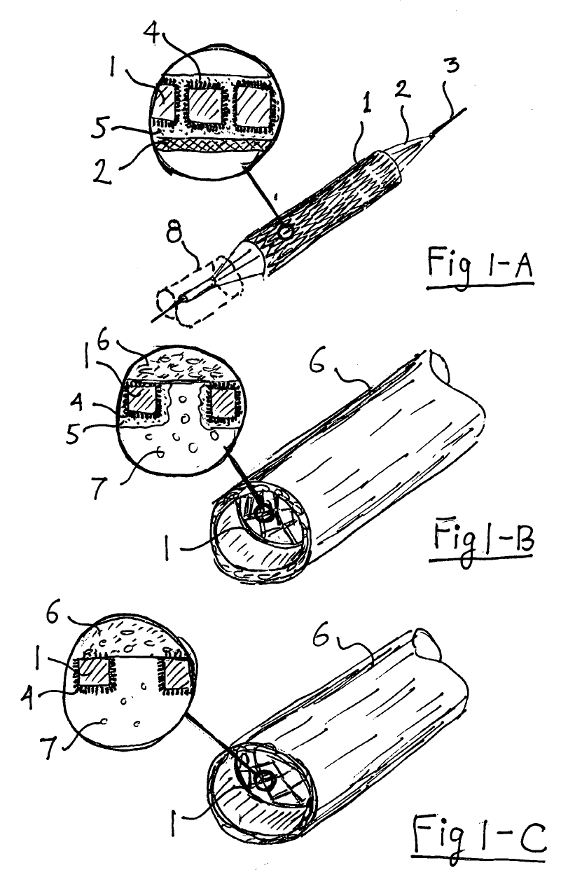

Stents are well known in cardiac medicine. Referring to FIG. 1A, a stent 1 is mounted on a balloon 2 typically guided by guide wire 3 and inserted into the artery by a catheter 8 in a percutaneous procedure. To avoid blood clotting caused by the stent and to avoid re-stenosis, the stent is coated by a superhydrophobic coating 4 (seen in the magnified cross section insert of FIG. 1A) and a protective coating 5. By the way of example the stent is made of type 316 stainless steel and the superhydrophobic coating is of the metallic type, formed in three simple steps:
1. Galvanically plate the stent (before mounting on balloon) with 1 um thickness of copper, over plated with a sub-micron layer of silver.
2. Form a superhydrophobic coating by dipping stent in AgNO₃ for a few minutes.
3. Treat the surface by dipping in HDFT (heptadecafluoro-1-decanathiol) and dry. For more details on forming the superhydrophobic treatment please refer to the paper: “Remarkably Simple Fabrication of Superhydrophobic Surfaces Using Electroless Galvanic Deposition” by I. A. Larmour, E. J. Bell and G. C. Saunders, Angewandte Chemie Int. Ed. 2007, 46, pages 1-4. This paper is hereby incorporated by reference. During this process the stent can be held by tweezers from the outside, as the outside surface will not be in direct contact with blood when deployed.

The protective coating is formed as following:
1. Prepare a near saturated solution of sucrose in water. Exact concentration is not important.
2. Add about 50% by volume of ethanol and mix. Exact amounts are not important.
3. Dip stent in solution for a few seconds and dry in warm air (40-50 degrees C.). Inspect coating under microscope. It should have a uniform glossy appearance. If some dull patches are visible re-coat by quickly dipping in solution and drying. Do not keep stent for more than a fraction of a second in coating solution otherwise existing coating will dissolve.
4. Mount stent on balloon. If it is desired to slow down the dissolution rate the coated and assembled stent can be dipped in a dilute solution of a blood compatible fat. The performance of the outside of the stent is not important, as after deployment only the inside area and the sidewalls of the stent structure are exposed to blood. These areas are protected from contact with blood until the stent is expanded and the balloon withdrawn, therefore a dissolution inhibitor should not be required for stents but may be required for items such as cardiac valves.

FIG. 1B shows the stent immediately after deployment in artery 6 and expansion by the balloon (balloon is removed). Protective sucrose coating 5 is just exposed to blood 7 and starts dissolving rapidly.

FIG. 1C shows the stent a few seconds later. Protective coat is dissolved and exposes the superhydrophobic surface 4 to blood 7.

Clearly the invention is not limited to any particular superhydrophobic coating and can be used to protect any sensitive coating. Also, while the preferred embodiment uses a sugar (glucose or sucrose) as a protective coating and alcohol (ethanol) as a wetting agent, any dissolvable solid compatible with the human body can be used as a protective coating and any wetting agent can be used. The wetting agent does not have to be mixed in the coating solution, it can be applied to the surface first, followed by the water based protective coating.

1. A method for introducing superhydrophobic articles into the body comprising the steps of:
coating the superhydrophobic article with a solid that can dissolve inside the body; and
inserting the coated article into the body.

2. A method as in claim 1 wherein said solid is a sugar.

3. A method as in claim 1 wherein said coating is done by dissolving said solid in water and adding a wetting agent to the solution.

4. A method as in claim 1 wherein said article is inserted into the blood circulation system.

5. A method as in claim 1 wherein said article is a stent with a superhydrophobic surface.

6. A method as in claim 1 wherein said article is a cardiac valve with a superhydrophobic surface.

7. A method as in claim 1 wherein said article is a pacemaker lead with a superhydrophobic surface.

8. A method as in claim 1 wherein said article is a pacemaker with a superhydrophobic surface.

9. A method as in claim 1 wherein said superhydrophobic article has a superhydrophobic surface a contact angle of over 170 degrees with water.

10. A method as in claim 1 wherein said coating is further treated to control the rate it dissolves in the body.

11. A water soluble protective coating applied to a superhydrophobic article in order to protect the superhydrophobic surface before article reached its final position inside the body.

12. A coating as in claim 11 containing a sugar.
